(19) United States
(12) Patent Application Publication
(10) Pub. No.: US 2025/0241838 A1
(43) Pub. Date: Jul. 31, 2025

(54) COSMETIC COMPOSITION FOR WITH ANTI-GLYCATION EFFECT FOR ENHANCING SKIN RESISTANCE TO AGING

(71) Applicant: L'OREAL, Paris (FR)

(72) Inventors: Ana DU, Clark, NJ (US); Noah WIEDER, Linden, NJ (US); Mark ZAW, Iselin, NJ (US); Matthew VOZZO, Cranford, NJ (US)

(73) Assignee: L'OREAL, Paris (FR)

(21) Appl. No.: 18/425,894

(22) Filed: Jan. 29, 2024

(51) Int. Cl. A61K 8/67; A61K 8/49; A61K 8/92; A61K 8/9789; A61Q 19/08

(52) U.S. Cl. CPC A61K 8/676; A61K 8/494; A61K 8/678; A61K 8/922; A61K 8/9789; A61Q 19/08; A61K 2800/48

(57) ABSTRACT

A cosmetic composition to combat skin aging, particularly on the neck, includes a blend of skin actives comprising vitamin C, a vitamin C derivative, or a combination thereof; dandelion root extract; and at least one additional active selected from the group consisting of caffeine, vitamin E, lavender oil, and combinations thereof; and an aqueous carrier system comprising water, wherein the cosmetic composition inhibits peptide and protein glycation. The skin actives are provided in an aqueous carrier in a single phase, biphase or emulsion wherein the cosmetic composition is a leave on treatment in the form of a cream, gel, lotion, toner, or serum.

### FIELD OF THE INVENTION

The present disclosure relates to a water-based cosmetic composition to combat skin aging that includes a blend of skin actives which inhibit peptide and protein glycation.

### BACKGROUND OF THE INVENTION

Skin aging and the attendant visible and textural changes to skin are a major focus of a wide variety of cosmetic products. Skin on the face and neck are particularly important targets for such treatments, especially skin on the neck which has different physical characteristics than skin on the face, and consequently shows aging in particular ways, which can include lines, wrinkles, crepiness, and sagging. Among skin cleansers, toners, moisturizers and other forms of treatment, a wide variety of skin care products provide blends of ingredients that address the signs of aging. While many ingredients in these products are known to have relevant biological effects, it remains a challenge to provide cosmetic treatments that include amounts and combinations of such ingredients sufficient to be effective.

One particular aspect of skin aging is the accumulation of advanced glycation end-products (sometimes referred to in the cosmetics literature as “AGEs”) in skin, which is implicated in the process of skin aging related to collagen degradation (correlated with loss of skin firmness) and melanogenesis (correlated with loss of skin tone evenness). Active ingredients with anti-glycation activity are known, including, for example, aminoguanidine (also known as gallic acid, extracted from hazelnut skins), alisin (extracted from garlic), and carnosine (also known as beta-alanyl-L-histidine, a dipeptide found in mammals); however, these actives are not always appropriate for all cosmetic vehicles due to ingredient incompatibility, cost, and negative impact on the cosmetic's aesthetics.

The present invention provides a novel combination of active ingredients showing a surprising anti-glycation effect that is synergistic in nature, wherein none of the individual active ingredients show anti-glycation activity at practical usage levels, yet the combination demonstrates a statistically significant inhibition of peptide and protein glycation.

### BRIEF SUMMARY OF THE INVENTION

In a first embodiment, the disclosure provides a cosmetic composition that includes a blend of skin actives comprising vitamin C, a vitamin C derivative, or a combination thereof; dandelion root extract; and at least one additional active selected from the group consisting of caffeine, vitamin E and derivatives thereof, lavender oil, and combinations thereof; and an aqueous carrier system comprising water, wherein the cosmetic composition inhibits peptide and protein glycation.

In some embodiments the vitamin C, vitamin C derivative, or combination thereof is present from about 0.2% to about 50%, dandelion root extract is present from about 0.05% to about 5%, and each one of the at least one additional active is present from about 0.01% to about 5%, all percentages by weight, based on the total weight of the cosmetic composition.

In some embodiments the vitamin C, vitamin C derivative, or combination thereof is present from about 0.2% to about 10%, dandelion root extract is present from about 0.08% to about 3%, and each one of the at least one additional active is present from about 0.01% to about 1%, all percentages by weight, based on the total weight of the cosmetic composition.

In some embodiments the at least one additional active comprises caffeine present from about 0.05% to about 0.5%, by weight, based on the total weight of the cosmetic composition.

In some embodiments the at least one additional active comprises vitamin E present from about 0.05% to about 0.5%, by weight, based on the total weight of the cosmetic composition.

In some embodiments the at least one additional active comprises lavender oil present from about 0.010% to about 0.020%, by weight, based on the total weight of the cosmetic composition.

In some embodiments the vitamin C, vitamin C derivative, or combination thereof is selected from the group consisting of l-ascorbic acid, acetyl-C (3-O-Ethyl ascorbic acid), magnesium ascorbyl phosphate, sodium ascorbyl phosphate, tetrahexyldecyl ascorbate, ascorbyl glucoside, and combinations thereof.

In some embodiments the vitamin C, vitamin C derivative, or combination thereof comprises acetyl C, and the at least one additional active comprises caffeine, vitamin E and derivatives thereof, and lavender oil.

In some embodiments the cosmetic composition is a single phase, biphase or emulsion, and wherein the cosmetic composition is a leave on treatment in the form of a cream, gel, lotion, toner, or serum.

In some embodiments the cosmetic composition includes at least one additive selected from the group consisting of solvents, surfactants, thickeners, oils, waxes, preservatives, brightening agents, effect pigments, essential oils, plant extracts, cosmetic actives, and combinations thereof.

In some embodiments the cosmetic composition includes: at least one water-soluble solvent selected from the group consisting of denatured alcohol, glycerin, propylene glycol, pentylene glycol, PEG/PPG/polybutylene glycol-8/5/3 glycerin, propanediol, butylene glycol, and combinations thereof; at least one thickener selected from the group consisting of hydroxyethylcellulose, xanthan gum, hydroxypropyl cellulose, sclerotium gum, guar gum, acacia gum, gellan gum, hydroxypropyl guar, and combinations thereof; at least one surfactant selected from the group consisting of PPG-6-decyltetradeceth-30, PEG-60 hydrogenated castor oil, laureth-23, polyglyceryl-2-oleate, polyglyceryl-4-caprate and combinations thereof; at least one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof.

In another embodiment, the disclosure provides a cosmetic composition that includes vitamin C, a vitamin C derivative, or a combination thereof; dandelion root extract; caffeine; vitamin E and derivatives thereof; and lavender oil; an aqueous carrier system comprising water and at least one additive selected from the group consisting of: at least one water-soluble solvent selected from the group consisting of denatured alcohol, glycerin, propylene glycol, PEG/PPG/polybutylene glycol-8/5/3 glycerin, pentylene glycol, propanediol, butylene glycol, and combinations thereof; at least one thickener including hydroxyethylcellulose; at least one surfactant selected from the group consisting of PPG-6-decyltetradeceth-30, PEG-60 hydrogenated castor oil, laureth-23, polyglyceryl-2-oleate, polyglyceryl-4-caprate and combinations thereof; at least one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof; and combinations thereof, wherein the cosmetic composition inhibits peptide and protein glycation, and wherein the cosmetic composition is a single phase, biphase or emulsion, and wherein the cosmetic composition is a leave on treatment in the form of a cream, gel, lotion, toner, or serum.

In some embodiments the vitamin C, a vitamin C derivative, or a combination thereof is acetyl-C.

In some embodiments acetyl C is present from about 0.2% to about 10% by weight, based on the total weight of the cosmetic composition; dandelion root extract is present from about 0.08% to about 3% by weight, based on the total weight of the cosmetic composition; caffeine is present from about 0.05% to about 1% by weight, based on the total weight of the cosmetic composition; vitamin E is present from about 0.05% to about 1% by weight, based on the total weight of the cosmetic composition; lavender oil is present from about 0.01% to about 1% by weight, based on the total weight of the cosmetic composition.

In some embodiments the at least one water-soluble solvent comprises alcohol, glycerin, propylene glycol, PEG/PPG/polybutylene glycol-8/5/3 glycerin, and pentylene glycol; the at least one thickener comprises hydroxyethylcellulose; the at least one surfactant comprises PPG-6-decyltetradeceth-30; the at least one additional active comprises adenosine, citric acid, hydroxypropyl tetrahydropyrantriol, methyldihydrojasmonate, phenoxyethanol, potassium hydroxide, salicylic acid, sodium citrate, and tetrasodium glutamate diacetate.

In some embodiments of the cosmetic composition acetyl C is present from about 0.2% to about 1.5%, dandelion root extract is present from about 0.08% to about 3%, caffeine is present from about 0.05% to about 0.5%, vitamin E is present from about 0.05% to about 0.5%, and lavender oil is present from about 0.010% to about 0.020%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

In some embodiments of the cosmetic composition acetyl C is present at about 1%, dandelion root extract is present at about 0.13%, caffeine is present at about 0.1%, vitamin E is present at about 0.1%, and lavender oil is present at about 0.015%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

In some embodiments of the cosmetic composition acetyl C is present at about 5%, dandelion root extract is present at about 0.22%, caffeine is present at about 0.5%, vitamin E is present at about 0.5%, and lavender oil is present at about 0.08%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

In some embodiments of the cosmetic composition acetyl C is present at about 10%, dandelion root extract is present at about 1.3%, caffeine is present at about 1%, vitamin E is present at about 1%, and lavender oil is present at about 0.15%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

In another embodiment, the disclosure provides a method for enhancing a keratinous substrate, the method including providing a leave-on, rinse-free cosmetic composition that includes: vitamin C, a vitamin C derivative, or a combination thereof; dandelion root extract; caffeine; vitamin E; lavender oil; and an aqueous carrier system comprising water; and optionally, an additive selected from the group consisting of: a water-soluble solvent selected from the group consisting of denatured alcohol, glycerin, propylene glycol, PEG/PPG/polybutylene glycol-8/5/3 glycerin, pentylene glycol, propanediol, butylene glycol, and combinations thereof; at least one thickener selected from the group consisting of hydroxyethylcellulose, xanthan gum, hydroxypropyl cellulose, sclerotium gum, guar gum, acacia gum, gellan gum, hydroxypropyl guar, and combinations thereof; a surfactant selected from the group consisting of PPG-6-decyltetradeceth-30, PEG-60 hydrogenated castor oil, laureth-23, polyglyceryl-2-oleate, polyglyceryl-4-caprate and combinations thereof; a one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof; and combinations thereof; applying the cosmetic composition to the keratinous substrate at least twice per day, for one or more days and in some examples for a period of at least 10 days; wherein the cosmetic composition inhibits peptide and protein glycation, and wherein the cosmetic composition is a single phase, biphase or emulsion, and wherein the cosmetic composition is a leave on treatment in the form of a cream, gel, lotion, toner, or serum.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

“Keratinous substrate” as used herein, includes, but is not limited to keratinous tissues, such as skin on the face and body, hair, and nails.

The disclosure relates to a cosmetic composition to combat skin aging includes a blend of skin actives comprising vitamin C, a vitamin C derivative, or a combination thereof; dandelion root extract; and at least one additional active selected from the group consisting of caffeine, vitamin E and derivatives thereof, lavender oil, and combinations thereof; and an aqueous carrier system comprising water, wherein the cosmetic composition inhibits peptide and protein glycation. The skin actives are provided in an aqueous carrier in a single phase, biphase or emulsion wherein the cosmetic composition is a leave on treatment in the form of a cream, gel, lotion, toner, or serum.

The cosmetic composition demonstrates a statistically significant inhibition of peptide and protein glycation as demonstrated in an in tubo (in vitro) experiments. Inasmuch as peptide and protein glycation is shown to impact the quality of skin and contributes to the signs of aging, the cosmetic composition is useful for addressing skin aging, including lines, wrinkles, crepiness, and sagging, wherein the benefits that may be conferred may include one or more of increasing firmness, increasing elasticity, improving hydration, and reducing the presence superficial adipose tissue.

In various embodiments, the cosmetic composition comprises the following ingredients and optional additional ingredients.

### Compositions

### Vitamin C/Derivatives

In various embodiments, the cosmetic composition includes vitamin C, a vitamin C derivative, or a combination thereof.

In some embodiments, the vitamin C, vitamin C derivative, or a combination thereof is selected from the group consisting of l-ascorbic acid, acetyl-C (3-O-Ethyl ascorbic acid), magnesium ascorbyl phosphate, sodium ascorbyl phosphate, tetrahexyldecyl ascorbate, ascorbyl glucoside, and combinations thereof.

In some embodiments, the vitamin C, vitamin C derivative, or a combination thereof comprises a vitamin C derivative that comprises acetyl-C (3-O-Ethyl ascorbic acid). In some embodiments, the vitamin C, vitamin C derivative, or a combination thereof comprises the vitamin C derivative that consists of acetyl-C (3-O-Ethyl ascorbic acid).

The cosmetic composition comprises the vitamin C, vitamin C derivative, or a combination thereof from about 0.2% to about 50%, or from about 0.2% to about 10%, or from about 0.5% to about 1.5%, or about 50%, or about 10%, or about 5%, or about 1%, or any suitable combination, sub-combination, range, or sub-range thereof by weight, based on the weight of the cosmetic composition. One of ordinary skill in the art, however, will appreciate that other ranges are within the scope of the invention.

Thus, vitamin C or vitamin C derivative, or a combination thereof is present by weight, based on the total weight of the cosmetic composition, from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, to about 50 percent, including increments and ranges therein and there between.

### Dandelion Root Extract

In various embodiments, the cosmetic composition includes dandelion root extract (TARAXACUM OFFICINALE (DANDELION) RHIZOME/ROOT EXTRACT). A commercially available example of dandelion root extract is APOLLUSKIN GRV—supplied by Silab.

The cosmetic composition comprises dandelion root extract from about 0.05% to about 50%, or from about 0.05% to about 5%, or from about 0.08% to about 3%, or from about 0.08% to about 1.5%, or from about 0.08% to about 0.18%, or about 0.13%, or about 0.22%, or about 1.3%, or about 2.2%, or about 3%, or any suitable combination, sub-combination, range, or sub-range thereof by weight, based on the weight of the cosmetic composition. One of ordinary skill in the art, however, will appreciate that other ranges are within the scope of the invention.

Thus, dandelion root extract is present by weight, based on the total weight of the cosmetic composition, from about 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, to about 50 percent, including increments and ranges therein and there between.

### Additional Actives

In various embodiments, the cosmetic composition includes at least one additional active selected from the group consisting of caffeine, vitamin E (and derivatives thereof, including tocopheryl acetate, tocopheryl glucoside, tocopheryl phosphate, tocopheryl succinate, alpha-, beta-, gamma-, and delta-tocopherol, and alpha-, beta-, gamma-, and delta-tocotrienol), lavender oil (LAVANDULA ANGUSTIFOLIA (LAVENDER) OIL), and combinations thereof. In some embodiments, the cosmetic composition includes as additional actives caffeine, vitamin E, and lavender oil.

In various embodiments, the cosmetic composition comprises each of the additional actives present in a range from about 0.01% to about 50%, or from about from about 0.01% to about 10%, or from about from about 0.01% to about 5%, or from about 0.01% to about 1%, or any suitable combination, sub-combination, range, or sub-range thereof by weight, based on the weight of the cosmetic composition.

In various embodiments, the cosmetic composition comprises caffeine, when present in a range from about 0.01% to about 50%, or from about from about 0.01% to about 10%, or from about from about 0.01% to about 5%, or from about 0.01% to about 1%, or from about 0.05% to about 1%, or from about 0.05% to about 0.5%, or about 0.1%, or about 0.5%, or about 1%, or from about or any suitable combination, sub-combination, range, or sub-range thereof by weight, based on the weight of the cosmetic composition.

In various embodiments, the cosmetic composition comprises vitamin E or a derivative thereof, when present in a range from about 0.01% to about 50%, or from about from about 0.01% to about 10%, or from about from about 0.01% to about 5%, or from about 0.01% to about 1%, or from about 0.05% to about 1%, or from about 0.05% to about 0.5%, or about 0.1%, or about 0.5%, or about 1%, or from about or any suitable combination, sub-combination, range, or sub-range thereof by weight, based on the weight of the cosmetic composition.

In various embodiments, the cosmetic composition comprises lavender oil, when present in a range from about 0.01% to about 50%, or from about from about 0.01% to about 10%, or from about from about 0.01% to about 5%, or from about 0.01% to about 1%, or from about 0.05% to about 0.5%, or from about 0.010% to about 0.020%, or about 0.015%, or about 0.08%, or about 0.15%, or any suitable combination, sub-combination, range, or sub-range thereof by weight, based on the weight of the cosmetic composition.

Thus, each one of the least one additional active, may be present by weight, based on the total weight of the cosmetic composition, from about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, to about 50 percent, including increments and ranges therein and there between

### Aqueous Carrier System

### Water

In various embodiments, the cosmetic composition includes water. The cosmetic composition comprises from about 1% to about 99% of water, or from about 10% to about 95%, or from about 15% to about 90%, or from about 40% to about 80%, or from about 45% to about 65%, or from about 55% to about 70%, or about 60% or any suitable combination, sub-combination, range, or sub-range thereof by weight, based on the weight of the cosmetic composition. One of ordinary skill in the art, however, will appreciate that other ranges are within the scope of the invention.

Thus, water is present by weight, based on the total weight of the cosmetic composition, from about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 to about 99 percent, including increments and ranges therein and there between.

The pH of the cosmetic composition is not limited but is generally greater than or equal % to about pH 6, and in some embodiments, is one of between 5 and 8, and between 5 and 7, and between 6 and 7, and in some embodiments is about 6. The pH can be adjusted to the desired value by addition of a base (organic or inorganic) to the cosmetic composition, for example ammonia or a primary, secondary, or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

### Water-Soluble Solvents

In some embodiments, the cosmetic composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), organic solvents, polyols, glycols, or mixtures thereof.

In some particular embodiments according to the disclosure, when present, a water-soluble solvent may include alcohol (denatured alcohol), glycerin, propylene glycol, and pentylene glycol, or combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In various embodiments the amount of the at least one water-soluble solvent, when present, is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one water soluble solvent, each water soluble solvent present in an amount as set forth herein above, wherein each different water soluble solvent may be present within one of the ranges selected from the ranges set forth herein above.

Thus, each one or combination of water-soluble solvents, when present, may be present by weight, based on the total weight of the cosmetic composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Additional Ingredients/Additives

In some embodiments, the cosmetic composition may include at least one additive selected from the group consisting of solvents, thickeners, surfactants, cosmetic actives, preservatives, oils, waxes, essential oils, plant extracts, pH adjusters, chelating agents, dispersants, brightening agents, pigments, effect pigments, dyes and colorants, powders, opacifiers, and combinations thereof.

In some particular embodiments, the cosmetic composition includes additives selected from the group consisting of a water-soluble solvent selected from the group consisting of denatured alcohol, glycerin, propylene glycol, PEG/PPG/polybutylene glycol-8/5/3 glycerin, pentylene glycol, propanediol, butylene glycol, and combinations thereof; at least one thickener selected from the group consisting of hydroxyethylcellulose, xanthan gum, hydroxypropyl cellulose, sclerotium gum, guar gum, acacia gum, gellan gum, hydroxypropyl guar, and combinations thereof; at least one surfactant selected from the group consisting of PPG-6-decyltetradeceth-30, PEG-60 hydrogenated castor oil, laureth-23, polyglyceryl-2-oleate, polyglyceryl-4-caprate and combinations thereof; at least one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof.

### Thickener

In some embodiments, the cosmetic composition may comprise one or more thickener. The thickener may be synthetic, mineral, or organic in nature, and may comprise a particulate of any shape.

In some embodiments according to the disclosure, when present, a thickener may be hydroxyethylcellulose.

More generally, by way of non-limiting example, thickeners may be chosen from xanthan gum, cellulose gum, acacia senegal gum, guar gum, sclerotium gum, dehydroxanthan gum, gellan gum, agar, algin, talc, mica, silica, silica surface-treated with a hydrophobic agent, fumed silica, kaolin, clay/smectite mineral (Argilla extract) polyamide (NYLON) powders (e.g. ORGASOL from Atochem), polyurethane powders, poly-β-alanine powder and polyethylene powder, powders of tetrafluoroethylene polymers (TEFLON), lauroyllysine, starch, boron nitride, hydrophobic silica aerogel particles, hollow polymer microspheres such as those of polyvinylidene chloride/acrylonitrile, for instance EXPANCEL (Nobel Industrie) or of acrylic acid copolymers (POLYTRAP from the company Dow Corning) and silicone resin microbeads (TOSPEARLS from Toshiba, for example), elastomeric polyorganosiloxane particles, precipitated calcium carbonate, magnesium carbonate, magnesium hydrogen carbonate, hydroxyapatite, hollow silica microspheres (SILICA BEADS from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate, magnesium myristate and combinations thereof.

In some embodiments, one or more thickener may be present in the cosmetic composition from about 0.01% to about 10%, or from about 0.01% to about 5%, or from about 0.01% to about 1%, or from about 0.01% to about 0.2%, or from about 0.01% to about 0.09%, or from about 0.02% to about 0.08%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one thickener, each thickener present in an amount as set forth herein above, wherein each different thickener may be present within one of the ranges selected from the ranges set forth herein above.

Thus, each one or combination of thickeners, when present, may be present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, to about 10 percent, including increments and ranges therein and there between.

### Surfactant

In some embodiments, the cosmetic composition may include one or more surfactants. In some particular embodiments, the cosmetic composition may comprise an emulsion that comprises one or more surfactants, alone or in combination, with one or more oil (as described below).

In some particular embodiments according to the disclosure, when present, a surfactant may include PPG-6-decyltetradeceth-30.

By way of non-limiting example, surfactants may be chosen from glyceryl stearate (for example, glyceryl stearate SE), stearic acid, potassium cetyl phosphate, and combinations thereof.

The one or more surfactant in the cosmetic composition may be present in an amount ranging from about 0.1% to about 5%, or from about 0.1% to about 1%, or from about 0.2% to about 0.8%, or from about 0.3% to about 0.5%. In at least certain exemplary embodiments, the surfactant is present in an amount less than about 1%, or less than about 0.8%. In some embodiments, the cosmetic composition includes more than one surfactant, each surfactant present in an amount as set forth herein above, wherein each different surfactant may be present within one of the ranges selected from the ranges set forth herein above.

Thus, each one or combination of surfactants, when present, may be present by weight, based on the total weight of the cosmetic composition, from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent, including increments and ranges therein and there between.

### Oil

In some embodiments, the cosmetic composition may include one or more oils. In some embodiments, the cosmetic composition may comprise an emulsion that comprises one or more oils, alone or in combination, with one or more surfactant (as described above).

In some embodiments, the cosmetic composition may include the essential oil Cymbopogon Martini Oil, or lavender oil (as an additional active), or a combination thereof.

As used herein, oil refers to any nonpolar compound that is a liquid at 25° C. and is hydrophobic and lipophilic. Oil that is suitable for use herein may be volatile or non-volatile. The term “volatile oil” relates to oil that is capable of evaporating on contact with the skin or a keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10-3 to 300 mmHg). The term “non-volatile oil” relates to oil which remains on the skin or the keratin fiber, at room temperature and atmospheric pressure, for at least several hours and which in particular has a vapor pressure of less than 10-3 mmHg (0.13 Pa).

In some embodiments, by way of non-limiting example, the one or more oil in the cosmetic composition may be chosen from botanical and essential oils, such as Helianthus annuus Seed Oil, Mentha piperita Oil, Rosmarinus officinalis (rosemary) Leaf Oil Pelargonium graveolens flower oil, Citrus aurantium dulcis (orange) peel oil, Menthe viridis (spearmint) leaf oil, Citrus aurantifolia (lime) oil, Melaleuca alternifolia (tea tree) leaf oil, Citrus grandis (grapefruit) peel oil, Citrus medica limonum (lemon) peel oil, rose flower oil, Eucalyptus globulus leaf oil, and combinations thereof.

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil, or a synthetic hydrocarbon-based oil. Further, suitable oil may be a mineral hydrocarbon-based oil, a plant hydrocarbon-based oil, or a synthetic hydrocarbon-based oil.

### Silicone Oils

The cosmetic composition may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

### Fluoro Oils

The cosmetic composition may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

### Hydrocarbon-Based Oils

The cosmetic composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cosmetic composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid, or linolenic acid;
(vii) carbonates, such as dicaprylyl carbonate, such as the product sold under the name Cetiol CC® by Cognis;
(viii) fatty amides, such as isopropyl N-lauroyl sarcosinate, such as the product sold under the trade name Eldew SL 205° from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the cosmetic composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the cosmetic composition may comprise one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol % to about 350 g/mol.

In some embodiments, the cosmetic composition may comprise polar emollients that include those derived from C12-C50 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

The one or more oil, when present, may be present in the cosmetic composition from about 0.0001% to about 20% or from about 0.001% to about 0.0010%, or from about 0.003% to about 0.004%, or from about 0.01% to about 0.1%, or from about 0.1% to about 10%, or from about 0.5% to about 20%, or from about 1% to about 10%, or from about 5% to about 10%, or from about 2% to about 7%, or from about 0.5% to about 2%, or from about 0.008% to about 0.01%, or from about 0.1% to about 0.2%, or from about 0.5% to about 2%, or from about or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one oil, each oil present in an amount as set forth herein above, wherein each different oil (such as, for example, plant oils and extracts with oils) may be present within one of the ranges selected from the ranges set forth herein above.

Thus, each one or combination of oils, when present, may be present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9 to about 10 percent, including increments and ranges therein and there between.

### Additional Cosmetic Actives

In some embodiments, the cosmetic composition may comprise one or more additional cosmetic actives in addition to vitamin C, dandelion root extract and one or more of caffeine, vitamin E and lavender oil as described herein. In some embodiments, more than one additional cosmetic active may be present in the cosmetic composition, the active selected to provide one or more benefits, including, but not limited to brightening/whitening, improvement of skin tone, blemish control or reduction, reduction of redness, anti-aging, and hydration. Some cosmetic activities may be antimicrobial or preservative in nature, or may include pH adjustors or chelating agents.

In some embodiments, the cosmetic composition includes at least one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, and combinations thereof.

In some particular embodiments, the cosmetic composition includes adenosine, citric acid, hydroxypropyl tetrahydropyrantriol, methyldihydrojasmonate, phenoxyethanol, potassium hydroxide, salicylic acid, sodium citrate, and tetrasodium glutamate diacetate.

In some embodiments, actives may be selected from anti-aging ingredients including hydroxypropyl tetrahydropyrantriol, one or more humectant, such as methyl gluceth-20, and one or more vitamin or vitamin derivative, or one of sodium citrate, citric acid, dipotassium glycyrrhizate, Anogeissus leiocarpus bark extract, hydrolyzed Rhodophycea extract, hydrolyzed linseed extract, Pseudoalteromonas ferment extract, Manilkara multinervis leaf extract, Lavandula hybrida oil, Grifola frondosa fruiting body extract, Plantago lanceolata leaf extract, Cyathea medullaris leaf extract, hydrolyzed hyaluronic acid, Malus domestica fruit cell culture extract, resveratrol, Salicyloyl Phytosphingosine, glaucine, retinol, retinoic acid, and combinations thereof.

In some embodiments, actives may be selected from oily skin treatment ingredients including lens esculenta (lentil) seed extract, sarcosine, Peumus boldus leaf extract, Crithmum maritimum extract, niacinamide, salicylic acid, hydroxyethylpiperazine ethane sulfonic acid, Spiraea ulmaria extract, pentaerythrityl tetra-di-t-butyl hydroxyhydrocinnamate and combinations thereof.

In some embodiments, actives may be selected from skin tone improving ingredients including Ophiopogon Japonicus root extract, Chenopodium quinoa seed extract, Tamarindus indica seed gum, xylitylglucoside (and) anhydroxylitol (and) xylitol, ceramide derivatives, Helianthus annuus (sunflower) seed oil unsaponifiables, myristyl malate phosphonic acid, mannose, 2-oleamido-I, 3-octadecanedi and combinations thereof.

In some embodiments, actives may be selected from anti-redness ingredients including madecassoside, saccharide isomerate, palmitoyl tripeptide-8, panthenol, Olea europaea (olive) leaf extract, Mentha piperita (peppermint) extract, Leontopodium alpinum extract, dipotassium glycyrrhizate, acetyl dipeptide-1 cetyl ester, acetyl tetrapeptide-15, Boswellia Serrata extract, sodium palmitoyl proline (and) Nymphaea alba flower extract and combinations thereof.

In some embodiments, actives may be selected from glycerin and glycols such as, for example, propylene glycol, butylene glycol, dipropylene glycol and diethylene glycol, glycol ethers such as monopropylene, dipropylene and tripropylene glycol alkyl(C1-C4) ethers, monoethylene, diethylene and triethylene glycol; salicylic acid and combinations thereof.

In some embodiments, actives may be selected from extracts of algae and of planktons, enzymes and coenzymes, ceramides, anti-oxidant compounds, including, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, curcumin, pinoresinol, hydroxytyrosol, cinnamic acid, caffeic acid, p-coumaric acid, baicalin, pine bark, and other extracts and combinations thereof.

In some embodiments, actives may be selected from anti-elastase and anti-collagenase agents, peptides, fatty acid derivatives, steroids; free-radical scavengers, vitamins, and vitamin derivatives, phenylethyl resorcinol, alpha and beta hydroxy acids, and hydrogen peroxide.

In some embodiments, actives may be selected from sunscreen agents (e.g., inorganic sunscreen agent, such as titanium dioxide and zinc oxide and organic sunscreen agents, such as octocrylene, ethylhexyl salicylate, homosalate, and avobenzone); and combinations thereof.

In some embodiments, one or more optional additional cosmetic active is water soluble.

Although the optional active additives are given as examples, it will be appreciated that other optional components compatible with cosmetic applications known in the art may be used.

In various embodiments, one or more additional cosmetic actives, alone or in combination, when present, can be present in the cosmetic composition from about 0.00001% to about 25%, or from about 0.00005% to about 15%, or from about 0.0001% to about 2%, or from about 0.05% to about 1%, or from about 0.02% to about 2%, or from about 0.2% to about 1%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition comprises two, three, four or more actives having some, or all of which may be water soluble. In some embodiments, the cosmetic composition includes more than one additional cosmetic active, each additional cosmetic active present in an amount as set forth herein above, wherein each different additional cosmetic active may be present within one of the ranges selected from the ranges set forth herein above.

Thus, each one or combination of additional cosmetic actives, when present, may be present by weight, based on the total weight of the cosmetic composition, from about 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, to about 25 percent, including increments and ranges therein and there between.

### Optional Additives

The cosmetic composition can also comprise one or more optional additive used in the cosmetics field which does not adversely affect the properties of the cosmetic composition according to the invention.

Optional additives may be selected from fragrances, pearlescent agents, fillers, powders, preservatives, proteins, protein hydrolysates, vitamins, silicones, odor absorbers and coloring materials; skin conditioners, hydrating agents, anti-microbial components, including, but not limited to, phenoxyethanol, chlorphenesin, capryloyl glycol and sodium salicylate; thickeners and fillers, fruit extracts, for example Pyrus malus (Apple) Fruit Extract, and Aloe Barbadensis Leaf Juice Powder; citric acid, sodium chloride; neutralizing or pH-adjusting agents (e.g., triethylamine (TEA) and sodium hydroxide), and combinations thereof.

In some embodiments, optional additives may be selected from fillers. The fillers may be of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores.

When present, a filler may include vinyl dimethicone/methicone silsesquioxane crosspolymer, ammonium polyacryloyldimethyl taurate, methyl methacrylate crosspolymer, silica silylate, mica, Zea may (corn) starch, magnesium oxide, nylon-12, nylon-66, cellulose, polyethylene, talc, talc (and) methicone, talc (and) dimethicone, perlite, sodium silicate, pumice, PTFE, Ammonium Polyacryloyldimethyl Taurate, polymethyl methacrylate, Oryza sativa (rice) starch, aluminum starch octenylsuccinate, potato starch modified, alumina, calcium sodium borosilicate, magnesium carbonate, hydrated dimethicone/vinyl dimethicone crosspolymer, sodium carboxymethyl starch, bismuth oxychloride, silica silylate, boron nitride, iron oxide, calcium carbonate, calcium sulfate (and) iron oxides, sodium potassium aluminum silicate, silica, silica (and) methicone, silica (and) dimethicone, polysilicone-22, polysilicone-8, polysilicone-11, methyl methacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer.

Although the optional active additives are given as examples, it will be appreciated that other optional components compatible with cosmetic applications known in the art may be used.

In various embodiments, when present, the amount of optional additives present in the cosmetic composition can range from about 0.001% to about 50%, or from about 0.01% to about 30%, or from about 1.5% to about 20%, and from about 5% to about 15%, or from about 0.005% to about 0.1%, or from about 0.05% to about 1%, or from about 0.06% to about 0.075%, or from about 0.1% to about 1%, or from about 0.25% to about 5%, and from about 0.5% to about 3.5%, or from about 0.2% to about 0.8%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one optional component, each optional component present in an amount as set forth herein above, wherein each different optional components may be present within one of the ranges selected from the ranges set forth herein above.

Thus, each one or combination of optional components, when present, may be present by weight, based on the total weight of the cosmetic composition, from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 25, 30, 35, 40, 45 to about 50 percent, including increments and ranges therein and there between.

### EXAMPLES

The following Examples are provided for illustrative purposes only and are not intended to be limiting.

### Example 1: Raw Materials

Raw materials used in the exemplified compositions include, in part, those listed in Table 1.

TABLE 1
Raw Materials (partial list)

| | % ACTIVE (IF LESS |
|---|---|
| RAW MATERIAL | THAN 100% |
| 3-O-ETHYL ASCORBIC ACID | |
| TARAXACUM OFFICINALE (DANDELION) | 4.3 (balance water, |
| RHIZOME/ROOT EXTRACT | pentylene glycol) |
| CAFFEINE | |
| TOCOPHERYL/TOCOPHERYL ACETATE | |
| LAVANDULA ANGUSTIFOLIA (LAVENDER) OIL | |
| HYDROXYPROPYL | 35 (balance water, |
| TETRAHYDROPYRANTRIOL | propylene glycol) |
| TETRASODIUM GLUTAMATE DIACETATE | 47.5 (balance water) |

### Example 2: Compositions

A representative example of an inventive composition is shown in Table 2.

TABLE 2
Inventive A (Amount in the table below reflects final wt % of active
in composition for those actives that are less than 100% in the RM)
Inventive A

| Ingredient | Amount |
|---|---|
| 3-O-ETHYL ASCORBIC ACID | 1.00 |
| ADENOSINE | 0.04 |
| ALCOHOL DENAT. | 3.00 |
| CAFFEINE | 0.10 |
| CITRIC ACID | 0.12 |
| CYMBOPOGON MARTINI OIL | 0.02 |
| GLYCERIN | 5.00 |
| HYDROXYETHYLCELLULOSE | 0.40 |
| HYDROXYPROPYL | 1.75 |
| TETRAHYDROPYRANTRIOL | |
| LAVANDULA ANGUSTIFOLIA | 0.015 |
| (LAVENDER) OIL | |
| METHYLDIHYDROJASMONATE | 0.10 |
| PEG/PPG/POLYBUTYLENE GLYCOL- | 2.00 |
| 8/5/3 GLYCERIN | |
| PHENOXYETHANOL | 0.50 |
| POTASSIUM HYDROXIDE | 0.18 |
| PPG-6-DECYLTETRADECETH-30 | 0.80 |
| PROPYLENE GLYCOL | 7.25 |
| PENTYLENE GLYCOL | 0.30 |
| SALICYLIC ACID | 0.45 |
| SODIUM CITRATE | 0.10 |
| TARAXACUM OFFICINALE | 0.13 |
| (DANDELION) RHIZOME/ROOT | |
| EXTRACT | |
| TETRASODIUM GLUTAMATE | 0.095 |
| DIACETATE | |
| TOCOPHEROL/TOCOPHERYL | 0.10 |
| ACETATE | |
| WATER | QS (~76) |

In some embodiments, cosmetic composition may include the following ingredients in amounts by total weight of the cosmetic composition that include: 3-O-ethyl ascorbic acid (^{˜}1.00), adenosine (^{˜}0.04), alcohol denat. (^{˜}3.00), caffeine (^{˜}0.10), citric acid (^{˜}0.12), Cymbopogon Martini oil (^{˜}0.02), glycerin (^{˜}5.00), hydroxyethylcellulose (^{˜}0.40), hydroxypropyl tetrahydropyrantriol (^{˜}1.75), Angustifolia lavandula (lavender) oil (^{˜}0.015), methyldihydrojasmonate (^{˜}0.10), PEG/PPG/polybutylene glycol-8/5/3 glycerin (^{˜}2.00), phenoxyethanol (^{˜}0.50), potassium hydroxide (^{˜}0.18), PPG-6-decyltetradeceth-30 (^{˜}0.80), propylene glycol (^{˜}7.25), pentylene glycol (^{˜}0.30), salicylic acid (^{˜}0.45), sodium citrate (^{˜}0.10), Taraxacum Officinale (dandelion) rhizome/root extract (^{˜}0.13), tetrasodium glutamate diacetate (^{˜}0.095), tocopherol/tocopheryl acetate (^{˜}0.10), water (^{˜}76), all present by weight based on the weight of the cosmetic composition.

### Example 2: In Vitro Glycation Study

A study was conducted in vitro/in tubo to evaluate if the ingredients of the inventive composition have anti-glycation effects in an in tubo environment.

The in tubo experiments were conducted to evaluate glycation of BSA (bovine serum albumin) under control conditions and with exposure to an embodiment of the inventive composition. Samples were prepared, including inventives, comparatives, baseline, and positive and negative controls.

A test plate was prepared with discrete wells for receiving each of the test compositions. Solutions representing the active ingredient concentrations listed under “Ingredient” in Table 3, below, were added to a well containing BSA+D-ribose (the Indicator Solution) and PBS. The total amount of Test Solutions added for the inventive compositions was always the same for all tests-25 microliters-5 microliters of each Test Solution (Ingredient) per well. PBS was used as the solvent for the Test Solution. Plates were prepared in triplicate.

Tested Compositions: Blank: PBS+BSA, Negative control: PBS+BSA+D-ribose, Positive control: PBS+BSA+D-ribose+Aminoguanidine, Five Inventive

The concentrations listed in the table below represent the concentration of active in the test solution added to each well—each ingredient other than the Indicator Solutions were added in 5 ml amounts. For example, referring to Inventive 4 in Table 3, five (5) microliters each of Activated C (3-O-ETHYL ASCORBIC ACID), Dandelion Extract, Caffeine, Lavender Oil, and Vitamin E (tocopherol), (5 microliters each->25 microliters total), each solution containing 50% of the active ingredient (Activated C, Dandelion Extract, Caffeine, Lavender Oil, and Vitamin E), wherein each Ingredient added had the initial concentration as shown in the column for Inventive 4, were added to wells containing the Indicator Solution. Inv 5 was tested the same way as Inv 4 except each Test Solution contained 100% of the active ingredient (except caffeine, as indicated in Table 3).

For Inventive compositions 1-5:5×5 microliters of each ingredients in the respective concentrations listed below were added to the [PBS+BSA+D-ribose] solution for a total volume of 250 microliter. In the 50% test combination, 2.5 microliter of each ingredients except caffeine were added Caffeine was separately diluted with 5 microliters of PBS to achieve a concentration of 1.5%. Then, the diluted caffeine was added to the remaining solution to make a total volume of 250 microliters. In the 100% test combination, 5 microliter of each ingredients except caffeine were added Caffeine was separately diluted with 5 microliters of PBS to achieve a concentration of 2%. Then, the diluted caffeine was added to the remaining solution to make a total volume of 250 microliters.

The samples evaluated in the glycation study are shown in Table 3. Amounts of ingredients listed in the Inventive compositions below represent amounts of Test Solution as described above, not final concentration of active ingredient.

TABLE 3
Samples for Glycation Study

| | Test Composition | | | | | | | |
|---|---|---|---|---|---|---|---|---|
| Ingredient | | Negative | Positive | | | | | |
| Ingredient | Baseline | Control | Control | Inv 1 | Inv 2 | Inv 3 | Inv 4 | Inv 5 |
| PBS | + | + | + | + | + | + | + | + |
| BSA | | + | + | + | + | + | + | + |
| D-Ribose | | + | + | + | + | + | + | + |
| Aminoguanidine | | | + | | | | | |
| Activated C (3-O- | | | | 1 | 5 | 10 | 50 | 100 |
| ETHYL ASCORBIC | | | | | | | | |
| ACID | | | | | | | | |
| Dandelion Extract | | | | 3 | 15 | 30 | 50 | 100 |
| Caffeine | | | | 0.1 | 0.5 | 1 | 1.5 | 2 |
| Lavender Oil | | | | 0.015 | 0.08 | 0.15 | 50 | 100 |
| Vitamin E | | | | 0.1 | 0.5 | 1 | 50 | 100 |
| (tocopherol) | | | | | | | | |

### Protocol:

Samples were plated in triplicate and readings taken at the indicated wavelength at day 1 and day 14.

Evaluations of Fluorescent Intensity were made at ex 370 nm/em 440 nm

Samples were evaluated at time intervals of 0 days, 3 days, 14 Days, 21 Days. The main purpose of incorporating time intervals is to provide sufficient duration for capturing the anti-glycation effect. It is crucial to build in multiple time points to adequately capture the anti-glycation effects since they can vary depending on the specific ingredients used.

Magnitude of fluorescence intensity is positively indicative of extent of glycation. Glycation is expected to be maximal with negative control (BSA plus D-ribose), while glycation of positive control with known antiglycation agent, Aminoguanidine, is expected to be reduced relative to negative control.

Summary Data from the glycation study are shown in Table 4 wherein the results presented were from day 14 readings.

TABLE 4
Summary Data

| | Fluorescence | Percent reduction |
|---|---|---|
| Test Composition | intensity | in glycation (%) |
| Baseline | — | — |
| Negative control | 3.805 | 0 |
| Positive control | 2.921 | 23 |
| Inv 1 | 3.134 | 17 |
| Inv 2 | 2.883 | 24 |
| Inv 3 | 2.939 | 23 |
| Inv 4 (50% all actives*) | 2.964 | 22 |
| Inv 5 (100% all actives*) | 2.664 | 30 |

*all actives includes Activated Vitamin C (3-O-Ethyl ascorbic acid), Dandelion Extract, Caffeine, Vitamin E (tocopherol), Lavender Extract

### Results:

Significant anti-glycation effect was observed with the inventive composition at all tested levels of components.

Evaluation of anti-glycation effect of individual ingredients: each of Activated C, Dandelion Extract, Caffeine, Lavender Oil, and Vitamin E (5 microliter quantities) were plated with 245 microliter of [PBS+BSA+D-ribose]. Concentrations of each ingredient added to the wells were as follows Activated C (100%, 50%, 10%, 5% and 1%), Dandelion Extract (100%, 50%, 30%, 15% and 3%), Caffeine (2%, 1.5%, 1%, 0.5% and 0.1%), Lavender Oil (100%, 50%, 0.15%, 0.08% and 0.15%), and Vitamin E (100%, 50%, 1%, 0.5% and 0.1%). Results with individual ingredients (not shown) demonstrated that each of Activated C and Dandelion Extract exhibited glycation effect only at high levels, and no glycation effect was observed with Caffeine, Lavender Oil, or Vitamin E.

Key ingredients with concentrations in the final formula showed significant benefits on glycation reduction compared to the D-ribose treated condition alone, demonstrating synergistic effects as a combination when no significant effects shown when each ingredient was tested as individually at the lower levels or final formula level.

The embodiments of the inventive composition showed significant benefits on glycation reduction compared to the negative control (D-ribose treated condition alone with no antiglycation agent), and as compared with the individual ingredients of the inventive composition when tested alone. Ascorbic acid and dandelion extract, respectively, when tested alone each demonstrated reduction of glycation at high doses only, while each of caffeine, lavender oil and vitamin E demonstrated no significant reduction in glycation.

The data reveal that embodiments of the inventive composition exhibit statistically less glycation than with actives alone, demonstrating synergistic effects as a combination, and overall results with inventive compositions are comparable to known antiglycation ingredient, aminoguanidine.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“One or more,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step, or material other than those specified in the claim and, in the latter instance, impurities ordinary associated with the specified material(s). The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

The terms “free” and “devoid” indicates that no reliably measurable excluded material is present in the cosmetic composition, typically 0% by weight, based on the total weight of the cosmetic composition. The term “essentially free” means that, while it prefers that no excluded material is present in the cosmetic composition, it is possible to have very small amounts of the excluded material in the cosmetic composition of the invention, provided that these amounts do not materially affect the advantageous properties of the cosmetic composition. In particular, “essentially free” means that excluded material can be present in the cosmetic composition at an amount of less than about 0.1% by weight, based on the total weight of the cosmetic composition.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9-11% and “about 2%” means 1.8-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1 to 10%, such as 2 to 8%, such as 3 to 5%,” is intended to encompass ranges of “1 to 8%,” “1 to 5%,” “2 to 10%,” and so on. All numbers, amounts, ranges, etc., are intended to be modified by the term “about,” whether or not so expressly stated. Similarly, a range given of “about 1 to 10%” is intended to have the term “about” modifying both the 1% and the 10% endpoints. Further, it is understood that when an amount of a component is given, it is intended to signify the amount of the active material unless otherwise stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to one or more embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

1. A cosmetic composition comprising:
a. a blend of skin actives comprising vitamin C, a vitamin C derivative, or a combination thereof; dandelion root extract; and at least one additional active selected from the group consisting of caffeine, vitamin E, lavender oil, and combinations thereof; and
b. an aqueous carrier system comprising water,
wherein the cosmetic composition inhibits peptide and protein glycation.

2. The cosmetic composition according to claim 1, wherein the vitamin C, vitamin C derivative, or combination thereof is present from about 0.2% to about 50%, dandelion root extract is present from about 0.05% to about 5%, and each one of the at least one additional active is present from about 0.01% to about 5%, all percentages by weight, based on the total weight of the cosmetic composition.

3. The cosmetic composition according to claim 1, wherein the vitamin C, vitamin C derivative, or combination thereof is present from about 0.2% to about 10%, dandelion root extract is present from about 0.08% to about 1.5%, and each one of the at least one additional active is present from about 0.01% to about 1%, all percentages by weight, based on the total weight of the cosmetic composition.

4. The cosmetic composition according to claim 1, wherein the at least one additional active comprises caffeine present from about 0.05% to about 0.5%, by weight, based on the total weight of the cosmetic composition.

5. The cosmetic composition according to claim 1, wherein the at least one additional active comprises vitamin E present from about 0.05% to about 0.5%, by weight, based on the total weight of the cosmetic composition.

6. The cosmetic composition according to claim 1, wherein the at least one additional active comprises lavender oil present from about 0.010% to about 0.020%, by weight, based on the total weight of the cosmetic composition.

7. The cosmetic composition according to claim 10, wherein the vitamin C, vitamin C derivative, or combination thereof is selected from the group consisting of 1-ascorbic acid, acetyl-C (3-O-Ethyl ascorbic acid), magnesium ascorbyl phosphate, sodium ascorbyl phosphate, tetrahexyldecyl ascorbate, ascorbyl glucoside, and combinations thereof.

8. The cosmetic composition according to claim 1, wherein the vitamin C, vitamin C derivative, or combination thereof comprises acetyl C, and wherein the at least one additional active comprises caffeine, vitamin E, and lavender oil.

9. The cosmetic composition according to claim 1, wherein the cosmetic composition is a single phase, biphase or emulsion, and wherein the cosmetic composition is a leave on treatment in the form of a cream, gel, lotion, toner, or serum.

10. The cosmetic composition according to claim 1, wherein the cosmetic composition includes at least one additive selected from the group consisting of solvents, surfactants, thickeners, oils, waxes, preservatives, brightening agents, effect pigments, essential oils, plant extracts, cosmetic actives, and combinations thereof.

11. The cosmetic composition according to claim 1, wherein the cosmetic composition comprises:
a. at least one water-soluble solvent selected from the group consisting of denatured alcohol, glycerin, propylene glycol, pentylene glycol, PEG/PPG/polybutylene glycol-8/5/3 glycerin, propanediol, butylene glycol, and combinations thereof;
b. at least one thickener selected from the group consisting of hydroxyethylcellulose, xanthan gum, hydroxypropyl cellulose, sclerotium gum, guar gum, acacia gum, gellan gum, hydroxypropyl guar, and combinations thereof;
c. at least one surfactant selected from the group consisting of PPG-6-decyltetradeceth-30, PEG-60 hydrogenated castor oil, laureth-23, polyglyceryl-2-oleate, polyglyceryl-4-caprate and combinations thereof;
d. at least one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof.

12. A cosmetic composition comprising:
a. vitamin C, a vitamin C derivative, or a combination thereof; dandelion root extract; caffeine; vitamin E; and lavender oil;
b. an aqueous carrier system comprising water and at least one additive selected from the group consisting of:
i. at least one water-soluble solvent selected from the group consisting of denatured alcohol, glycerin, propylene glycol, pentylene glycol, PEG/PPG/polybutylene glycol-8/5/3 glycerin, propanediol, butylene glycol, and combinations thereof;
ii. at least one thickener selected from the group consisting of hydroxyethylcellulose, xanthan gum, hydroxypropyl cellulose, sclerotium gum, guar gum, acacia gum, gellan gum, hydroxypropyl guar, and combinations thereof;
iii. at least one surfactant selected from the group consisting of PPG-6-decyltetradeceth-30, PEG-60 hydrogenated castor oil, laureth-23, polyglyceryl-2-oleate, polyglyceryl-4-caprate and combinations thereof;
iv. at least one cosmetic active selected from the group consisting of adenosine, acid, citric hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof;
v. combinations thereof,
wherein the cosmetic composition inhibits peptide and protein glycation, and
wherein the cosmetic composition is a single phase, biphase or emulsion, and wherein the cosmetic composition is a leave on treatment in the form of a cream, gel, lotion, toner, or serum.

13. The cosmetic composition according to claim 1, wherein the vitamin C, a vitamin C derivative, or a combination thereof is acetyl-C.

14. The cosmetic composition according to claim 13, wherein
a. acetyl C is present from about 0.5% to about 10% by weight, based on the total weight of the cosmetic composition;
b. dandelion root extract is present from about 0.08% to about 1.5% by weight, based on the total weight of the cosmetic composition;
c. caffeine is present from about 0.05% to about 1% by weight, based on the total weight of the cosmetic composition;
d. vitamin E is present from about 0.05% to about 1% by weight, based on the total weight of the cosmetic composition;
e. lavender oil is present from about 0.01% to about 1% by weight, based on the total weight of the cosmetic composition.

15. The cosmetic composition according to claim 14, wherein the at least one water-soluble solvent comprises alcohol, glycerin, propylene glycol, pentylene glycol and PEG/PPG/polybutylene glycol-8/5/3 glycerin; the at least one thickener comprises hydroxyethylcellulose; the at least one surfactant comprises PPG-6-decyltetradeceth-30; the at least one additional active comprises adenosine, citric acid, hydroxypropyl tetrahydropyrantriol, methyldihydrojasmonate, phenoxyethanol, potassium hydroxide, salicylic acid, sodium citrate, and tetrasodium glutamate diacetate.

16. The cosmetic composition according to claim 15, wherein acetyl C is present from about 0.05% to about 1.5%, dandelion root extract is present from about 0.08% to about 0.18%, caffeine is present from about 0.05% to about 0.5%, vitamin E is present from about 0.05% to about 0.5%, and lavender oil is present from about 0.010% to about 0.020%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

17. The cosmetic composition according to claim 15, wherein acetyl C is present at about 1%, dandelion root extract is present at about 0.13%, caffeine is present at about 0.1%, vitamin E is present at about 0.1%, and lavender oil is present at about 0.015%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

18. The cosmetic composition according to claim 15, wherein acetyl C is present at about 5%, dandelion root extract is present at about 0.22%, caffeine is present at about 0.5%, vitamin E is present at about 0.5%, and lavender oil is present at about 0.08%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

19. The cosmetic composition according to claim 15, wherein acetyl C is present at about 10%, dandelion root extract is present at about 1.3%, caffeine is present at about 1%, vitamin E is present at about 1%, and lavender oil is present at about 0.15%, all percentages by weight, based on the total weight of the cosmetic composition, and wherein the cosmetic composition is provided as an emulsion and is a leave on treatment in the form of a cream, gel, lotion, toner, or serum, and wherein the cosmetic composition inhibits peptide and protein glycation.

20. A method for enhancing a keratinous substrate, comprising:
a. providing a leave-on, rinse-free cosmetic composition comprising:
i. vitamin C, a vitamin C derivative, or a combination thereof; dandelion root extract; caffeine; vitamin E; lavender oil; and an aqueous carrier system comprising water; and
ii. optionally, an additive selected from the group consisting of:
1. a water-soluble solvent selected from the group consisting of denatured alcohol, glycerin, propylene glycol, pentylene glycol, PEG/PPG/polybutylene glycol-8/5/3 glycerin, propanediol, butylene glycol, and combinations thereof;
2. a thickener selected from the group consisting of hydroxyethylcellulose, xanthan gum, hydroxypropyl cellulose, sclerotium gum, guar gum, acacia gum, gellan gum, hydroxypropyl guar, and combinations thereof;
3. a surfactant selected from the group consisting of PPG-6-decyltetradeceth-30, PEG-60 hydrogenated castor oil, laureth-23, polyglyceryl-2-oleate, polyglyceryl-4-caprate and combinations thereof;
4. a one cosmetic active selected from the group consisting of adenosine, citric acid, hydroxypropyl tetrahydropyrantriol, methyldihydrojasmonate, phenoxyethanol, potassium hydroxide, salicylic acid, sodium citrate, tetrasodium glutamate diacetate, peptides, niacinamide, carnosine, and combinations thereof;
5. combinations thereof,
b. Applying the cosmetic composition to the keratinous substrate at least twice per day;
wherein the cosmetic composition inhibits peptide and protein glycation, and wherein the cosmetic composition is a single phase, biphase or emulsion, and wherein the cosmetic composition is a leave on treatment in the form of a cream, gel, lotion, toner, or serum.
